(19) United States
(12) Patent Application Publication
(10) Pub. No.: US 2018/0028763 A1
(43) Pub. Date: Feb. 1, 2018

(54) NEEDLE FOR DRUG INJECTION AND DRUG INJECTION APPARATUS COMPRISING THE SAME

(71) Applicants: Joon-Shik SHIN, Seoul (KR); Jinho LEE, Seoul (KR); In-Hyuk HA, Seoul (KR)

(72) Inventors: Joon-Shik SHIN, Seoul (KR); Jinho LEE, Seoul (KR); In-Hyuk HA, Seoul (KR)

(21) Appl. No.: 15/551,343

(22) PCT Filed: Feb. 16, 2016
(86) PCT No.: PCT/KR2016/001540

§ 371 Date: Aug. 16, 2017

(30) Foreign Application Priority Data
Feb. 16, 2015 (KR) 10-2015-0023207

(51) Int. Cl. A61M 5/32; A61M 5/20; A61M 5/315

(52) U.S. Cl. CPC A61M 5/3291; A61M 5/3287; A61M 5/31513; A61M 5/20; A61M 5/3286; A61M 2205/584

(57) ABSTRACT

A needle for drug injection, and a drug injection apparatus comprising the same are disclosed. The needle for drug injection is formed in the shape of a cylinder, of which one end comprises a needle tip and the other end comprises; an needle body configured to be opened so that drug can be injected thereinto; and at least one outlet through which the drug is discharged, the outlet being provided on one side of the needle body, wherein the outlet is formed to have an inclination angle in the direction from the one end to the other end.

### BACKGROUND OF THE INVENTION

### 1. Field of the Invention

The present invention relates to a needle for drug injection, more precisely a needle for drug injection and a drug injection apparatus comprising the same

### 2. Description of the Related Art

Meridian muscles have been studied continuously for a long time from the past till today. For example, studies have been made including the meridian muscle structure, meridian muscle and the movement, theoretical conceptual analysis of meridian muscle, diagnosis of meridian muscle disease, treatment of meridian muscle disease, and extended concept of meridian muscle, etc.

Herein, original meaning of the “muscle” includes muscle, tendon, superficial vein, and nerve in <Yellow Emperor's Inner Canon>. In <Meridian Muscles>, the muscle means muscle and tendon. According to the domestic experimental research papers of anatomical studies on the meridian muscles, the meaning includes each meridian muscle, nerve, and blood vessel. More detailed analysis results were obtained by dividing the muscles into a thin layer, a middle layer and a deep layer.

That is, meridian muscles play an important role in bending/stretching of joint and movement of limbs, and such actions induced by meridian muscles include the movement of muscle, nerve, and blood vessels as well.

In general, the meridian system and the meridian muscle of human body show abnormal activity or malfunction or disorder when damage is caused by external stimuli or because of internal metabolism imbalance, resulting in pain or body inequality in patients because those symptoms can affect other organs too.

To eliminate the cause of body inequality and to increase natural healing potential and restoring force of human body, a needle is intentionally used. In particular, a method to stimulate acupuncture points or meridian system and meridian muscle by using an electric needle through which electricity flows has been widely used. At this time, the point for needle injection, in order to stimulate meridian system and meridian muscle, is called the acupuncture point.

Among needles, some needles are designed for drug injection after stimulating acupuncture points of human body. Since needles do not have scales, we don't know how deeply the needle is injected in, it is very difficult to inject drug accurately in the damaged meridian muscle tissues (muscle, nerve, blood vessel, and joint, etc).

With the conventional needle, it is difficult to insert the needle in the meridian muscle tissue more than a certain angle when the point area for injection has many structures such as bones. Even if the needle is possibly injected, it is still difficult to deliver drug to the side of the lesion through which the needle passes.

To solve the problems above, it is required to develop a novel needle that is useful for drug injection.

Korean Patent No. 10-1439466 relates to the device and kit for drug injection, wherein the space for drug storage contains a mixing enhancer for increasing the mixing of a drug and a solvent. However, the device for drug injection only works to discharge drug only through the tip of the needle so that a large amount of drug is hardly delivered.

That is, in the case of the needle, since the drug can be ejected only forward in the area having many bonds, it is difficult to deliver the drug to the lesion.

As an effort to supplement the problem above, Korean utility model gazette No. 20-0412054 describes a supply device of medicine needle designed to maximize the treatment effect by providing acupuncture treatment and drug injection simultaneously in the acupuncture point in Oriental medicine. This device comprises an needle body with one side of the pipe polished through which drug can be injected as contacted with the acupuncture point of human body and which is also useful for acupuncture therapy; and an acupuncture bottle useable as connected to a general syringe.

However, the polished part of the needle body has an inclined plane on the needle tip, through which drug can spills, suggesting that it is hard to deliver drug precisely to the target lesion.

### SUMMARY OF THE INVENTION

It is an object of the present invention to provide a needle for drug injection to deliver a large amount of drug to a lesion accurately with minimizing tissue damage, and a drug injection apparatus comprising the same.

It is another object of the present invention to provide a needle for drug injection wherein a plurality of holes are formed so as to spray drug over a large area and control the spraying direction to inject drug at a desired position by a user, and a drug injection apparatus comprising the same.

It is also an object of the present invention to provide a needle for drug injection designed to discharge drug according to a predetermined dose with the regulated ejection intensity, and a drug injection apparatus comprising the same.

To achieve the above objects, the needle for drug injection of the present invention is formed in the shape of a cylinder wherein one end comprises a sharp needle tip and the other end comprises: an needle body configured to be opened so that drug can be injected thereinto; and at least one outlet through which the drug is discharged, the outlet being provided on one side of the needle body, wherein the outlet is formed to have an inclination angle in the direction from the one end to the other end.

The needle for drug injection of the present invention can additionally include a location indicator formed on the outer surface of the needle body in the same direction with the outlet to indicate the location of the outlet.

The needle for drug injection of the present invention can additionally include a scale on the outer surface of the needle body at predetermined intervals.

The needle body contains an inner wall on which the outlet is formed and a drug injection hole into which the drug is loaded inside the inner wall above.

One end of the drug injection hole is formed in the shape of a hemisphere and the other end is opened. The thickness of the hole is 0.4˜0.6 mm.

The distance between the outer surface of the needle tip and the inner surface of the inner wall is 0.1˜0.5 mm.

The outlet is inclined at the angle of 30˜60° in the direction from one end of the needle body to the other end, which characteristically passes through the inner wall in the width of 0.5˜0.2 mm in diameter.

The needle tip is formed in a conical shape and the angle of the conical shape is 30˜120°. The length in the longitudinal direction is 0.1˜0.5 cm.

The length of the needle body in the longitudinal direction is 1˜10 cm and the diameter is 0.5˜1 mm.

The scale is formed at regular intervals of 0.5˜2 cm to confirm the depth of the needle body.

### Advantageous Effect

The needle for drug injection of the present invention and the drug injection apparatus comprising the same can deliver a large amount of drug to a lesion accurately with minimizing tissue damage.

Also, multiple holes are formed on the needle for drug injection of the present invention so as to discharge drug in a large area and control the discharge direction.

The discharge of the drug can be controlled according to the predetermined dose and the ejection intensity can also be controlled.

### BRIEF DESCRIPTION OF THE DRAWINGS

The application of the preferred embodiments of the present invention is best understood with reference to the accompanying drawings, wherein:

FIG. 1a and FIG. 1b are perspective views illustrating the drug injection apparatus according to a preferred embodiment of the present invention.

FIG. 2a and FIG. 2b are exemplary views illustrating the needle for drug injection according to a preferred embodiment of the present invention.

FIG. 3a and FIG. 3b are exemplary views illustrating the drug discharge by the needle for drug injection according to a preferred embodiment of the present invention.

FIG. 4 is an exemplary view illustrating the instruction of using the needle for drug injection in the case of a spinal disease according to a preferred embodiment of the present invention.

FIG. 5 is an exemplary view illustrating the instruction of using the needle for drug injection in the case of a hip disease according to a preferred embodiment of the present invention.

FIG. 6 is an exemplary view illustrating the instruction of using the needle for drug injection in the case of a knee disease according to a preferred embodiment of the present invention.

FIG. 7 is an exemplary view illustrating the instruction of using the needle for drug injection in the case of Achilles tendinitis according to a preferred embodiment of the present invention.

FIG. 8 is an exemplary view illustrating the instruction of using the needle for drug injection in the case of a shoulder disease according to a preferred embodiment of the present invention.

FIG. 9 is an exemplary view illustrating the instruction of using the needle for drug injection in the case of nerve entrapment syndrome according to a preferred embodiment of the present invention.

### DESCRIPTION OF THE PREFERRED EMBODIMENTS

Hereinafter, the preferred embodiments of the present invention are illustrated in more detail with the attached figures. First, when reference marks are given to each component presented in each figure, the same marks are given to the same components even though they are separately presented in different figures. In the description of the present invention, a detailed explanation of known functions and configurations incorporated herein can be omitted when it may make the subject matter of the present invention unclear.

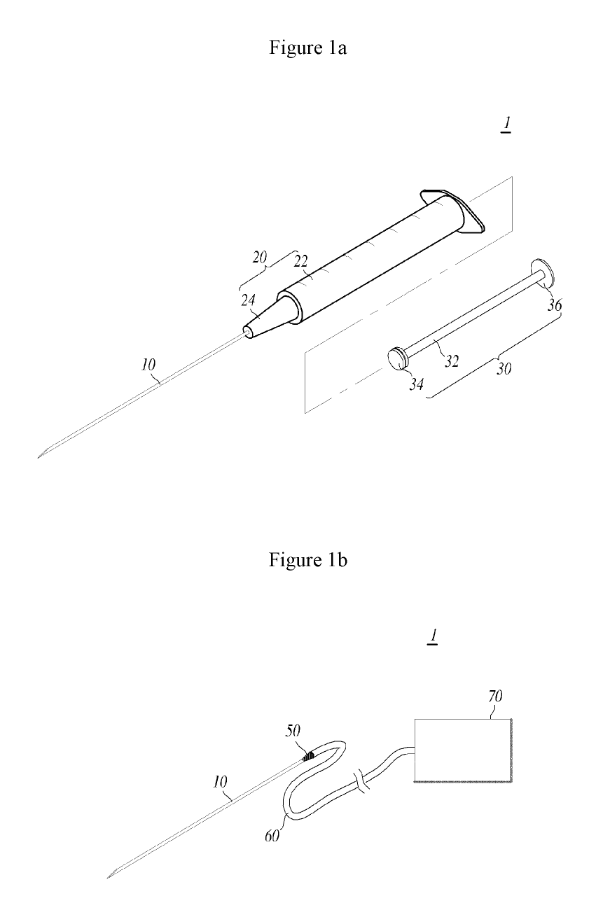

FIG. 1a and FIG. 1b are perspective views illustrating the drug injection apparatus according to a preferred embodiment of the present invention. FIG. 1a is a diagram illustrating the manual drug injection apparatus, and FIG. 1b is a diagram illustrating the automatic drug injection apparatus.

As shown in FIGS. 1a and 1b, the drug injection apparatus (1) delivers the drug manually or automatically to a lesion through the needle for drug injection (10) inserted in the treatment site.

In the case of a manual drug injection apparatus, the drug injection apparatus (1) includes a needle for drug injection (10), a needle loader (20), and a drug injector (30).

The needle for drug injection (10) contains at least one outlet through which the drug can be discharged when it is inserted in the treatment site. One end of the needle for drug injection (10) comprises a needle tip and the other end is opened for drug injection. In particular, the outlet can be formed on one side of the drug injection needle (10) to discharge the drug to the side.

Therefore, the needle for drug injection (10) is efficient in minimizing tissue damage in the treatment site and locating the treatment site without being exposed to radioactive materials. The needle for drug injection (10) can also deliver the drug into a lesion laterally by approaching from the side of the treatment site instead of inserting directly in the treatment site particularly when the treatment site is hard to approach.

The needle loader (20) is to insert and fix the needle for drug injection (10) and to store the drug loaded therein. The needle loader (20) includes a body part (22) formed in a pipe shape and storing drug and a needle fixing part (24) for fixing the needle for drug injection (10).

The body part (22) includes a drug injector (30) which would be described hereinafter. At this time, the body part (22) includes a scale to check the amount of drug inserted. The needle fixing part (24) is to connect the needle for drug injection (10) and the body part (22).

The drug injector (30) is to inject drug through the needle for drug injection (10) by giving the pressure on the drug stored in the needle loader (20). The drug injector (30) includes a piston (32), a rubber member (34), and a pressing part (36).

The piston (32) is formed in a columnar shape between the rubber member (34) and the pressing part (36). A rubber member (32) is inserted in one end of the piston (32) and a pressing part (36) having a larger diameter than the piston (32) is formed in the other end of the piston (32).

One end of the drug injector (30) with the rubber member (34) inserted therein is inserted into the needle loader (20) to apply pressure to the drug. Through this, the drug is injected into the needle for drug injection (10). At this time, the rubber member (34) prevents leakage from occurring while the needle loader (20) and the drug injector (30) are inserted.

In the case of an automatic drug injection apparatus, the drug injection apparatus (1) includes a needle for drug injection (10), a needle loader (20), a tube (60), and a digital injector (70).

The needle for drug injection (10) is the same as the needle for drug injection (10) used in the manual drug injection apparatus. The needle loader (50) is functioning equally to the needle loader (20) used in the manual drug injection apparatus but is directly connected to the tube (60) with eliminating the body part (22).

One end of the tube (60) is connected to the needle loader (50) and the other end is connected to the digital injector (70). At this time, the needle loader (50) can be integrally connected to the tube (60), but if necessary it can be arranged as separated.

That is, the tube (60) plays a role of transporting the drug injected from the digital injector (70) to the needle loader (50).

The digital injector (70) injects the drug into the tube (60) according to the predetermined control signal. The digital injector (70) can set the drug injection time, injection amount, and injection strength. Therefore, the digital injector (70) can inject the drug in various injection patterns.

The drug injection apparatus (1) shown in FIGS. 1a and 1b is only one example, but it is not always limited thereto.

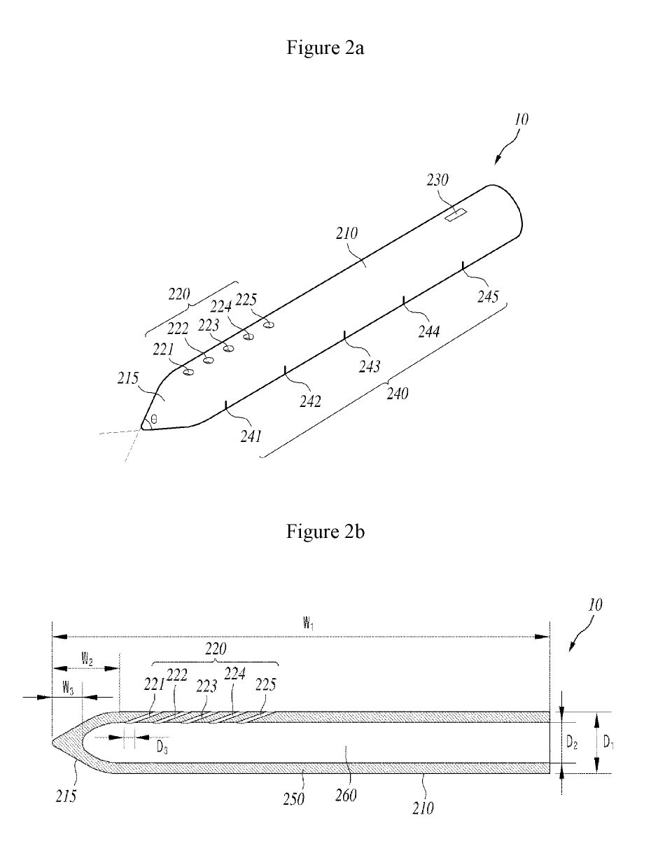

FIG. 2a and FIG. 2b are exemplary views illustrating the needle for drug injection according to a preferred embodiment of the present invention. FIG. 2a is a perspective view of the needle for drug injection. FIG. 2b is a sectional view of the needle for drug injection.

As shown in FIGS. 2a and 2b, the needle for drug injection (10) contains an needle body (210), an outlet (220), a location indicator (230), and a scale (240).

The needle body (210) is a body of the needle for drug injection (10), which is formed in the shape of a cylinder. The needle body (210) has a length (W₁) in the longitudinal direction of 1 to 10 cm and a diameter (D₁) of 0.5 to 1 mm. At this time, the length and the diameter of the needle body (210) can be varied according to the purpose of use and the treatment site.

One end of the needle body (210) comprises a needle tip (215). The needle tip (215) is the part where the drug injection needle (10) is first inserted into the treatment site. Therefore, the needle tip (215) is formed in a conical shape to facilitate the insertion. The angle of the conical shape is preferably 30˜120° and the angle can be varied according to the use and the treatment site. The needle tip (215) has a length (W₂) in the longitudinal direction of 0.1 to 0.5 cm.

The other end of the needle body (210) is opened, through which the drug is injected. That is, the other end of the needle body is connected to the needle fixing part (24, 25) to inject the drug. The needle body (210) includes an inner wall (250) and includes a drug injection hole (260) into which the drug is injected.

The inner wall (250) is formed within the needle body (210) and the needle tip (215) and has a thickness of 0.05 to 0.2 mm. Preferably, the thickness is 0.1 mm. In particular, the distance (W₃) between the inner peripheral surface of the inner wall (250) and the outer peripheral surface of the needle tip (215) is 0.1˜0.5 mm. The outlet (220) which will be described hereinafter is formed on one side of the inner wall (250) and the drug injection hole (260) is formed on the inner wall (250).

The drug injection hole (260) is a hole through which the drug injected from the needle fixing part (24, 54) is transferred to the outlet (220). In particular, one end of the drug injection hole (260) is formed in a hemispherical shape not to let the drug deposited and instead to push out the drug easily, and the other end is opened through which the drug is injected. The diameter (D₂) of the drug injection hole (260) is 0.4˜0.6 mm.

The outlet (220) is at least one, and can be formed more than one arranged in a line at regular intervals, for example from the first outlet to the fifth outlet (221, 222, 223, 224, and 225). Herein, the arrangement of the outlet (220) is not limited to a line and can be achieved in any form that is useful for letting out the drug more easily at the treatment site.

The outlet (220) is to pass through counter-clockwise from the center axis of the needle body (210) in the inclination angle of 30˜60°. In addition, the outlet (220) penetrates the inner wall (250) with the diameter (D₃) of 0.05˜0.2 mm. At this time, the outlet angle and the diameter of the penetrated hole can be changed according to the use and the treatment site.

The location indicator (230) is formed on the outer surface of the needle body (210) in the same direction as the outlet (220) to indicate the location of the outlet (220). At this time, the location indicator (230) presents a color image on the outer surface for the indication. That is, when the needle for drug injection (10) is injected in the treatment site and thus the outlet (220) is not seen, the location indicator (230) indicates the location of the outlet (220) to a user, by which the user can control the direction of drug discharge. Herein, the location indicator (230) can also be formed in the needle loader (20, 50) so that a user can more easily grasp the location of the outlet (220).

The scale (240) indicates the depth at which the needle for drug injection (10) is inserted into the treatment site. The scale (240) forms the first to fifth scales (241, 242, 243, 244, and 245) on the outer surface of the needle body (210) at predetermined intervals to provide the depth to of the needle insertion. The predetermined interval is preferably 0.5˜2 cm and more preferably 1 cm.

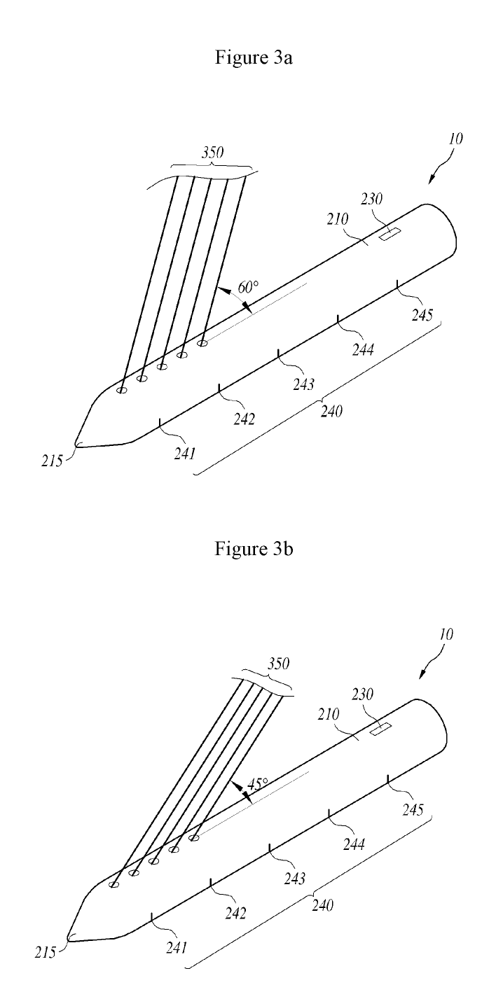

FIG. 3a and FIG. 3b are exemplary views illustrating the drug discharge by the needle for drug injection according to a preferred embodiment of the present invention. FIG. 3a is a diagram illustrating the drug discharge from the outlet having the penetration angle of 60°. FIG. 3b is a diagram illustrating the drug discharge from the outlet having the penetration angle of 45°.

As shown in FIGS. 3a and 3b, a drug can be discharged to a preferred target area by regulating the penetration angle of the outlet (230) of the needle for drug injection (10). That is, the needle for drug injection (10) can adjust the inclination angle of the outlets from the first outlet to the fifth outlet (230) for the penetration in the range of 45°˜60° in order to regulate the direction of the drug discharge (350).

The outlet (230) is formed on one side of the needle body (210) to discharge the drug from the side of the needle body (210). At this time, the outlet (230), passing through the needle body (210), has the inclination angle in the direction toward the location indicator (250) from the needle tip (215). The inclination angle is between 30°˜60°.

When the inclination angle is in the range above, the drug discharge (350) is prevented from spilling down instead of being delivered to a lesion. That is, the pressure to project the drug in the beginning is lower than the set pressure. Therefore, if the inclination angle of the outlet (230) is not allowed, the drug would spill down. On the other hand, if the outlet (230) has the inclination angle in the range above, the drug would not spill down until the initial pressure reaches the set pressure and once the pressure reaches the set pressure the drug is discharged (350) and delivered to a lesion.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Experimental Example 1: Instruction of Using the Needle for Drug Injection in the Case of a Spinal Disease

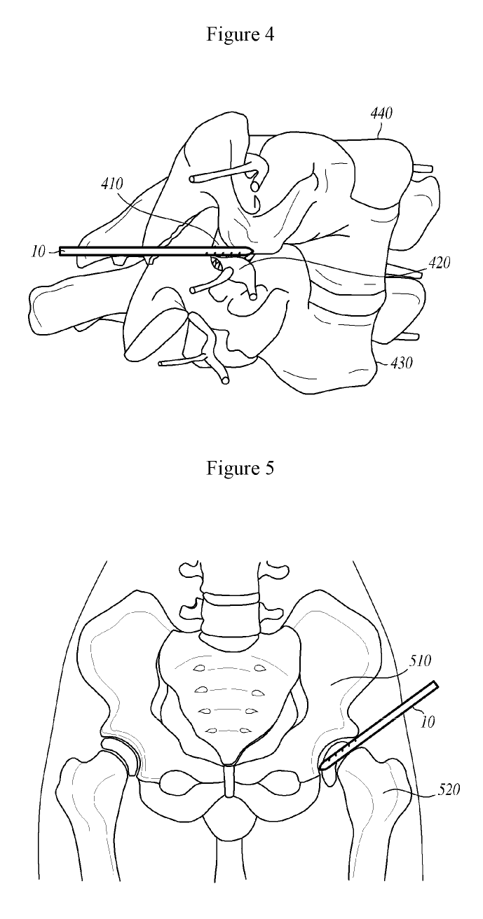

FIG. 4 is an exemplary view illustrating the instruction of using the needle for drug injection in the case of a spinal disease according to a preferred embodiment of the present invention.

As shown in FIG. 4, the needle for drug injection (10) can be used for spinal disease. For the treatment of spinal disease, the drug should be injected as close as possible to the nerve compression site between the bone (430) and the spinal disc (440). The spinal disease herein includes cervical disc, thoracic disc, lumbar disc, stenosis, anterior displacement, and degenerative disc, etc.

The drug injection needle (10) is inserted in the direction of nerve entrapment (420) in which the posterior joint is degenerated (410). That is, when the direction of the treatment site is located horizontally, the needle for drug injection (10) is inserted horizontally and can spray the drug on the side. In particular, when the drug is discharged, inflammation region might be washed as well.

Therefore, the needle for drug injection (10) can locate a treatment site without the guidance of real-time X-ray or CT and can minimize nerve damage in order to discharge the drug.

### Experimental Example 2: Instruction of Using the Needle for Drug Injection in the Case of a Hip Disease

FIG. 5 is an exemplary view illustrating the instruction of using the needle for drug injection in the case of a hip disease according to a preferred embodiment of the present invention.

As shown in FIG. 5, the needle for drug injection (10) can be used for hip disease. The hip disease herein includes degenerative hip arthritis, avascular necrosis of the femoral head, hip impingement syndrome, and hip pain, etc. The hip is a closed structure, and has many bony structures (bones), which makes the insertion of the needle with an accurate angle difficult. If the hip tissue is damaged in the course of the needle insertion, the symptoms of the patient may be severe and recovery can be delayed.

The needle for drug injection (10) is inserted with the needle tip sliding alongside the bone by using the joint bone (510) and the femur (520) as markers and then discharges the drug on the side accurately and safely.

### Experimental Example 3: Instruction of Using the Needle for Drug Injection in the Case of a Knee Disease

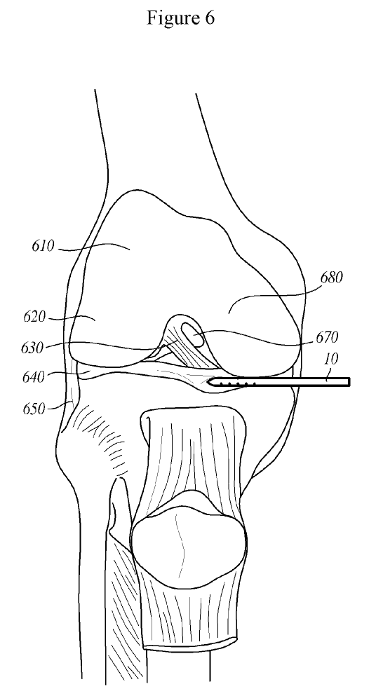

FIG. 6 is an exemplary view illustrating the instruction of using the needle for drug injection in the case of a knee disease according to a preferred embodiment of the present invention.

As shown in FIG. 6, the needle for drug injection (10) can be used for knee disease. The knee disease herein includes degenerative knee arthritis, arthritis, and ligament injury, etc. The knee joint has a structure in which a bone called patella (not shown) surrounds the front part, under which a knee surface (610), lateral femoral epicondyle (620), anterior cruciate ligament (630), lateral meniscus (640), collateral ligament (650), knee ligament (660), posterior cruciate ligament (670), and medial epicondyle (680) are located.

The conventional needle cannot deliver the drug to a target area under the patella which barricades therein. However, the needle for drug injection (10) of the invention can discharge a large amount of drug more accurately to the treatment site by adjusting the outlet direction toward the articular cavity after the needle is inserted under the patella.

### Experimental Example 4: Instruction of Using the Needle for Drug Injection in the Case of Achilles Tendinitis

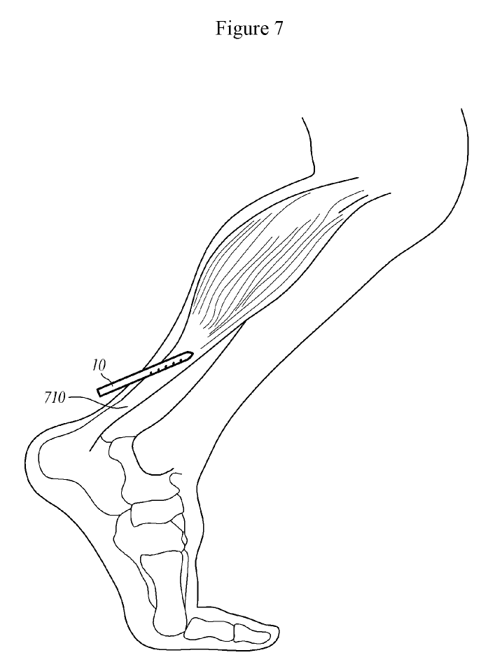

FIG. 7 is an exemplary view illustrating the instruction of using the needle for drug injection in the case of Achilles tendinitis according to a preferred embodiment of the present invention.

As shown in FIG. 7, the needle for drug injection (10) can be used for Achilles tendinitis. Achilles tendinitis occurs in the inferior area (710) that faces the bone, not the outside.

The conventional needle is inserted from the outside with tearing the Achilles tendon and discharges the drug therein. On the other hand, the needle for drug injection (10) of the present invention is inserted from the side of the Achilles tendon and discharges the drug sideway toward the inside of the Achilles tendon, by which the needle for drug injection (10) of the invention can discharge the drug accurately to the damaged area.

### Experimental Example 5: Instruction of Using the Needle for Drug Injection in the Case of a Shoulder Disease

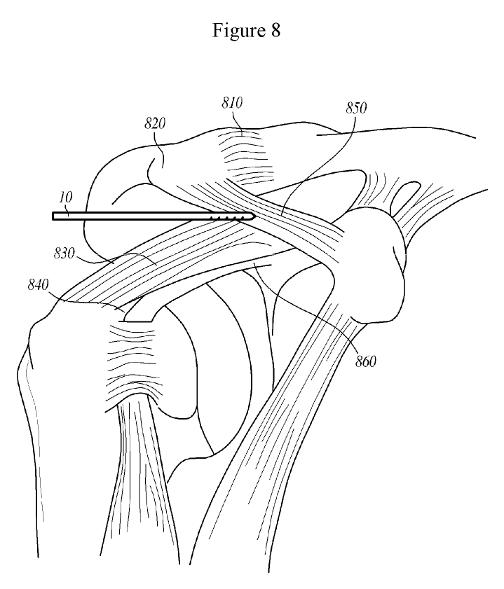

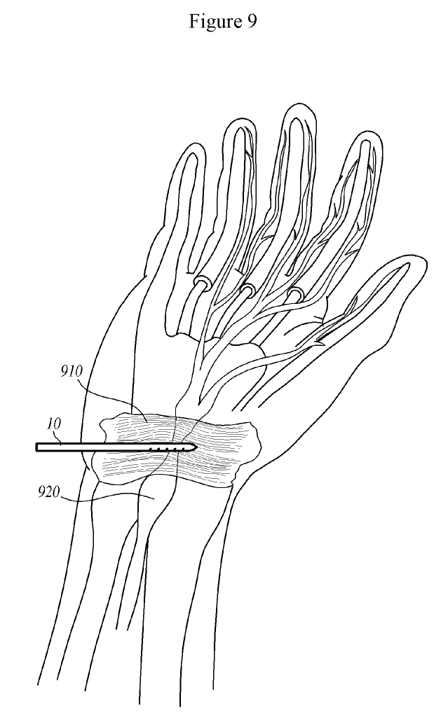

FIG. 8 is an exemplary view illustrating the instruction of using the needle for drug injection in the case of a shoulder disease according to a preferred embodiment of the present invention.

As shown in FIG. 7, the needle for drug injection (10) can be used for shoulder disease. The shoulder disease herein includes supraspinatus tendinitis, impingement syndrome, frozen shoulder, and arthritis, etc. In the shoulder joint, the acromioclavicular joint (810), the acromion (820), the biceps tendon (840), the coracoacromial ligament (850), and the starting point of the biceps long head tendon (860) are located, so the space is narrow and the structures (bones) are plenty.

Therefore, in the case of supraspinatus tendinitis which is a common disease, there seems no angle to insert the needle because of the acromion (820). However, the needle for drug injection (10) can be inserted horizontally under the acromion (820) to discharge the drug in the direction toward the supraspinatus tendinitis (830).

### Experimental Example 6: Instruction of Using the Needle for Drug Injection in the Case of Nerve Entrapment Syndrome

FIG. 9 is an exemplary view illustrating the instruction of using the needle for drug injection in the case of nerve entrapment syndrome according to a preferred embodiment of the present invention.

As shown in FIG. 9, the needle for drug injection (10) can be used for nerve entrapment syndrome. Nerve entrapment syndrome is caused when an area is given a lot of pressure due to inflammation of the ligaments so as to press the peripheral nerves, and includes all the symptoms such as paresthesia in the neural distribution area, abnormal sense, pain, and muscle weakness. The nerve entrapment syndrome herein includes ulnar nerve palsy, radial nerve palsy, and carpal tunnel syndrome, etc. In particular, in the case of nerve entrapment syndrome, the treatment site is the neuron pathway area, so when the needle for drug injection (10) is inserted, attention is required not to cause any nerve injury.

The conventional needle is inserted vertically, so that the lateral carpal ligament (910) and the median nerve (920) might be damaged. On the other hand, the needle for drug injection (10) of the invention can be inserted horizontally in parallel with the median nerve (910) and then discharge the drug sideway toward the median nerve (910).

### BRIEF DESCRIPTION OF THE MARK OF DRAWINGS

1: drug injection apparatus
10: needle for drug injection
20, 50: needle loader
22: body part
24: needle fixing part
30: drug injector
32: piston
34, 65: rubber member
36: pressing part
60: tube
70: digital injector
210: needle body
215: needle tip
220: outlet
221: 1^{st} outlet
222: 2^{nd} outlet
223: 3^{rd} outlet
224: 4^{th} outlet
222: 5^{th} outlet
230: location indicator
240: scale
241: 1^{st} scale
242: 2^{nd} scale
243: 3^{rd} scale
244: 4^{th} scale
245: 5^{th} scale
250: inner wall
260: drug injection hole
350: drug discharge
410: degenerated posterior joint
420: nerve entrapment
430: bone
440: spinal disc
510: joint bone
520: femur
610: knee surface
620: lateral femoral epicondyle
630: anterior cruciate ligament
640: lateral meniscus
650: collateral ligament
660: knee ligament
670: posterior cruciate ligament
680: medial epicondyle
710: Achilles tendon
810: acromioclavicular joint
820: acromion
830: supraspinatus tendinitis
840: biceps tendon
850: coracoacromial ligament
860: starting point of biceps long head tendon
910: lateral carpal ligament
920: median nerve

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A needle for drug injection characteristically composed of a needle body formed in the shape of a cylinder wherein one end comprises a needle tip and the other end is opened through which drug is injected; and at least one outlet formed on one side of the needle body through which drug is discharged and at this time the outlet is formed to have an inclination angle in the direction from the one end to the other end.

2. The needle for drug injection according to claim 1, wherein the needle additionally includes a location indicator to indicate the location of the outlet which is formed on the outer surface of the needle body in the same direction as the outlet.

3. The needle for drug injection according to claim 1, wherein the needle additionally includes a scale formed on the outer surface of the needle body at predetermined intervals.

4. The needle for drug injection according to claim 1, wherein the needle contains an inner wall on which the outlet is formed and a drug injection hole into which the drug is loaded inside the inner wall.

5. The needle for drug injection according to claim 4, wherein one end of the drug injection hole is formed in the shape of a hemisphere and the other end is opened, and the thickness of the hole is 0.4˜0.6 mm.

6. The needle for drug injection according to claim 4, wherein the distance between the outer surface of the needle tip and the inner surface of the inner wall is 0.1˜0.5 mm.

7. The needle for drug injection according to claim 4, wherein the outlet is inclined at the angle of 30˜60° in the direction from one end of the needle body to the other end, which characteristically passes through the inner wall in the width of 0.5˜0.2 mm in diameter.

8. The needle for drug injection according to claim 1, wherein the needle tip is formed in a conical shape and the angle of the conical shape is 30˜120°, and the length in the longitudinal direction is 0.1˜0.5 cm.

9. The needle for drug injection according to claim 1, wherein the length of the needle body in the longitudinal direction is 1˜10 cm and the diameter is 0.5˜1 mm.

10. The needle for drug injection according to claim 3, wherein the scale is formed at regular intervals of 0.5˜2 cm in order to confirm the depth of the needle body insertion.

11. A drug injection apparatus comprising the needle for drug injection according to claim 1.

12. The drug injection apparatus according to claim 11, wherein the apparatus additionally includes a needle loader for inserting the needle for drug injection and fixing the inserted needle for drug injection.

13. The drug injection apparatus according to claim 12, wherein the needle loader includes a body part where the drug is stored.

14. The drug injection apparatus according to claim 13, wherein the apparatus additionally includes a drug injector comprising a piston in the shape of a column wherein a rubber member is inserted in one end of the piston and the said end is inserted in the needle loader to provide the contained drug in the needle for drug injection.

15. The drug injection apparatus according to claim 12, wherein the apparatus additionally includes a tube which is connected to the needle loader to deliver drug and a digital injector which is connected to the tube to discharge drug according to the predetermined control signal.
